(19) United States
(12) Patent Application Publication
(10) Pub. No.: US 2024/0399070 A1
(43) Pub. Date: Dec. 5, 2024

(54) ENDOSCOPIC PUNCTURE NEEDLE UNIT

(71) Applicant: The University of Tokyo, Tokyo (JP)

(72) Inventor: Tomoki TODO, Tokyo (JP)

(21) Appl. No.: 18/804,706

(22) Filed: Aug. 14, 2024

Related U.S. Application Data
(63) Continuation of application No. PCT/JP2023/004882, filed on Feb. 14, 2023.

(30) Foreign Application Priority Data
Feb. 17, 2022 (JP) 2022-022965

(51) Int. Cl. A61M 5/32

(52) U.S. Cl. CPC A61M 5/3202; A61M 5/3287; A61M 2202/206

(57) ABSTRACT

An endoscopic puncture needle unit is an endoscopic puncture needle unit for administering a virus formulation to a tumor site, including a sheath to be inserted through a forceps channel of an endoscope, a cannula to be inserted into the sheath and having a distal end portion processed into a puncture needle, and a grip member configured to support a sheath proximal end portion which is a proximal end portion of the sheath and a cannula proximal end portion which is a proximal end portion of the cannula, a syringe configured to hold the virus formulation being attachable to the grip member. The cannula is formed integrally from an attachment portion to be directly attached to a connection base of the syringe to the puncture needle configured to discharge the virus formulation.

This application is a bypass continuation of International Patent Application PCT/JP2023/004882, filed on Feb. 14, 2023, which claims benefit of priority from Japanese Patent Application 2022-022965, filed on Feb. 17, 2022, the contents of both of which are incorporated herein by reference.

### TECHNICAL FIELD

The present invention relates to an endoscopic puncture needle unit for administering a virus formulation to a tumor site.

### BACKGROUND ART

The inventors of the present invention are engaged in research and development of a virus therapy of altering the genome of herpes simplex virus (HSV) by means of genetic engineering to produce, as a virus for cancer therapy, genetically modified HSV that grows only in cancer cells and does not damage normal tissues, and directly destroying cancer cells with this virus for cancer therapy.

More specifically describing, the virus therapy is a method for infecting cancer (tumor) cells with a propagative virus for cancer therapy to cure a cancer by means of a direct cell-killing effect of the virus itself associated with viral replication. The propagative virus for cancer therapy contrived through genetic modification to selectively replicate only in cancer cells replicates when infecting cancer cells and kills host cancer cells in the process of replication. The replicated virus for cancer therapy scatters to the surroundings to infect the cancer cells again, and then repeat replication, cell death, and infection to develop an antitumor effect. On the other hand, the virus for cancer therapy having infected normal cells does not replicate, so that the normal tissues are not damaged. Viral growth in a tumor induces specific antitumor immunity. Consequently, the direct cell-killing effect obtained by viral replication and triggering of the specific antitumor immunity enable the cancer to be cured without expression of a particular therapeutic gene, and a high efficacy can be expected as a new cancer therapeutic method.

Thus, in order to apply the virus therapy to a therapy of a brain tumor, the inventors of the present invention have developed a needle device for conducting a stereotactic surgery in a brain, biopsying an affected area, and administering a virus formulation containing a virus for cancer therapy to the affected area (see, for example, Patent Literature 1).

### CITATION LIST

### Patent Literature

Patent Literature 1: Japanese Patent Laid-Open No. 2012-80943

### SUMMARY OF INVENTION

### Technical Problem

In common endoscopic puncture needle units including the above-described needle device, a space exists between a proximal end portion of a cannula that carries a drug solution and a syringe that supplies the drug solution. Specifically, the space is an inner space of a connecting portion that connects the cannula and accepts the syringe, and the drug solution supplied from the syringe is required to once fill the inner space until reaching an inner cavity of the cannula. In the common puncture needle units, the puncture needle and the cannula are formed separately and the cannula is molded by a resin tube in some cases. In such cases, however, an inner cavity of the tube is increased, which requires a considerably larger amount of a drug solution than a dosage to be administered to an affected area. In the research and development of the virus therapy being advanced by the inventors of the present invention, the virus formulation to be administered to a tumor site is very rare and expensive, and the virus contained in the formulation is infectious and different in adhesiveness from common pharmaceuticals. It is therefore demanded to reduce a residual amount to be left in the puncture needle unit as much as possible and reduce an adsorbed amount in the inner cavity of the cannula as much as possible to bring a drug solution amount of a drug solution containing a required virus formulation as close as possible to a dosage to be administered to a tumor site.

The present invention has been made to solve the problems and provides an endoscopic puncture needle unit that enables a planned dosage to be appropriately administered to a tumor site without wasting a rare and expensive virus formulation.

### Solution to Problem

An endoscopic puncture needle unit in an aspect of the present invention is a puncture needle unit for administering a virus formulation to a tumor site, including a sheath to be inserted through a forceps channel of an endoscope, a cannula to be inserted into the sheath and having a distal end portion processed into a puncture needle, and a grip member configured to support a sheath proximal end portion which is a proximal end portion of the sheath and a cannula proximal end portion which is a proximal end portion of the cannula, a syringe configured to hold the virus formulation being attachable to the grip member. The cannula is formed integrally from an attachment portion to be directly attached to a connection base of the syringe to the puncture needle configured to discharge the virus formulation. Since the endoscopic puncture needle unit thus configured can reduce an inner cavity of the sheath in diameter and save the conventionally required space between the syringe and the cannula, a residual amount of the virus formulation to be left in the puncture needle unit can be reduced as much as possible.

In the above-described endoscopic puncture needle unit, the cannula has an inner cavity diameter of more than or equal to 0.2 mm and less than or equal to 1.0 mm, and preferably has a total length of more than or equal to 1.5 m and less than or equal to 2.6 m in a case where the puncture needle unit is used in conjunction with a gastrointestinal endoscope, for example, and preferably has a total length of more than or equal to 0.6 m and less than or equal to 1.0 m in a case where the puncture needle unit is used in conjunction with a bronchoscope. The inventors of the present invention have gained knowledge that these conditions should only be satisfied as a result of repeated trial and error so as to achieve in a balanced manner the respective requests for reduction of the drug solution amount to be left in the inner cavity of the cannula as much as possible, suitability to the application of insertion through the forceps channel of the endoscope, and smooth administration of the drug solution from the puncture needle in response to an operation of a surgeon.

In the above-described endoscopic puncture needle unit, a material of the cannula may be stainless steel. By forming the cannula of stainless steel, even a virus having higher adhesiveness than adhesiveness of common pharmaceuticals is less likely to be adsorbed onto the inner cavity, which contributes to reduction of a required amount of the virus formulation. At this time, a material of the sheath may be fluororesin. The sheath, when formed of fluororesin, is easily inserted through the forceps channel of the endoscope, and the cannula, when formed of stainless steel, is easily reduced in diameter of the inner cavity and has excellent flexibility. Such a combination of materials is also favorable in terms of slidability in a case where the cannula moves forward/backward in the sheath.

In the above-described endoscopic puncture needle unit, the grip member may have a fixing section configured to support the sheath proximal end portion and a movable section configured to support the cannula proximal end portion and to be capable of moving forward/backward with respect to the fixing section in an insertion direction of the cannula into the sheath, and the puncture needle may be configured to protrude from an open distal end portion of the sheath or to be stored in the open distal end portion when the movable section is moved forward/backward with respect to the fixing section. Such a configuration can store the puncture needle to prevent the puncture needle from being caught when being inserted through the forceps channel of the endoscope, and can prevent other tissues from being damaged by causing the puncture needle to protrude when approaching a tumor site, for example. At this time, the grip member may be configured to have a protruding amount adjustment mechanism configured to change in a stepwise manner a protruding amount of the puncture needle configured to protrude from the open distal end portion. When the protruding amount can be changed in a stepwise manner, a surgeon operating the puncture needle unit is able to intuitively recognize the protruding amount of the puncture needle. The puncture needle may be formed to be curved with respect to the insertion direction into the sheath. By curving the puncture needle, the cannula is easily inserted through the sheath.

### Advantageous Effect of Invention

According to the present invention, an endoscopic puncture needle unit that enables a planned dosage to be appropriately administered to a tumor site without wasting a rare and expensive virus formulation can be provided.

### BRIEF DESCRIPTION OF DRAWINGS

FIG. 1 is a front view of a puncture needle unit according to the present embodiment.

FIG. 2 is a cross-sectional view of the puncture needle unit.

FIG. 3 FIGS. 3A-3C are diagrams describing forward/backward movement of a puncture needle in response to an operation of a grip member.

### DESCRIPTION OF EMBODIMENT

An embodiment of the present invention will be described with reference to the accompanied drawings. Note that in the respective drawings, components denoted by the same reference character have the same or similar configuration. In the respective drawings, in a case where a plurality of structures having the same or similar configuration exist, some are denoted by reference characters and denoting others by the same reference characters is omitted in some cases so as to avoid complicatedness.

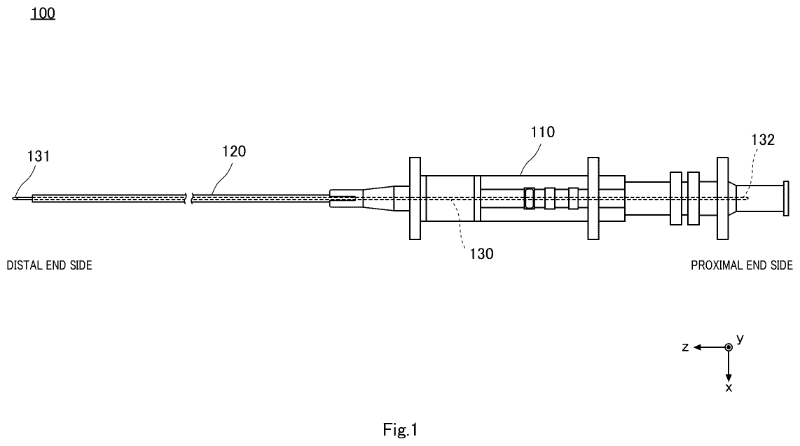

FIG. 1 is a front view of an endoscopic puncture needle unit 100 according to the present embodiment. The puncture needle unit 100 according to the present embodiment is assumed to be used for, in a virus therapy of infecting cancer cells with a virus for cancer therapy contained in a virus formulation and curing a cancer by means of a cell-killing effect of the virus, administering the virus formulation to a target tumor site.

The puncture needle unit 100 is mainly formed from a grip member 110, a sheath 120, and a cannula 130. The grip member 110 is a grasping section to be grasped by a surgeon. The grip member 110 presents a pencil shape as a whole and has some flanges for finger catch or the like. The grip member 110 is formed of polycarbonate resin, for example.

The sheath 120 is an outer cylinder tube extending from a distal end of the grip member 110 to be inserted through a forceps channel of an endoscope. The sheath 120 is formed of fluororesin, for example. Since fluororesin commonly has a small friction coefficient, the surgeon is able to easily insert the sheath 120 through the forceps channel of the endoscope. In the present embodiment, the sheath 120 has an outer diameter of 1.46 mm and an inner diameter of 0.86 mm.

The cannula 130 is an inner cylinder tube to be inserted into the sheath 120 and has an inner cavity bearing a function of carrying a virus formulation. The cannula 130 is formed of stainless steel, for example. A stainless steel material slides well on fluororesin, so that the cannula 130 is easily inserted through an inner cavity of the sheath 120, and the inserted cannula 130 is easily moved forward/backward. In a case where the outer diameter is reduced, the cannula 130 has excellent flexibility and favorably follows a curve of the sheath 120. Since the stainless steel material is also excellent in workability, it is easy to reduce the inner cavity. In the present embodiment, the cannula 130 has an outer diameter of 0.55 mm and an inner diameter of 0.38 mm.

In the cannula 130, a distal end portion to be delivered to a tumor site is processed into a puncture needle 131. The puncture needle 131 discharges the virus formulation carried through the inner cavity of the cannula 130. In the cannula 130, an end on the proximal end side opposite to the distal end side on which the puncture needle 131 is provided functions as an attachment portion 132 to be directly attached to a connection base of a syringe which will be described later. The attachment portion 132 may be subjected to end-face processing so as to be easily attached to the connection base of the syringe and may be provided with a cap.

In FIG. 1, portions of the cannula 130 located inside the sheath 120 and inside the grip member 110 are indicated by dotted lines. As is also understood from the drawing, the cannula 130 is formed integrally from the distal end side processed into the puncture needle 131 to the proximal end side functioning as the attachment portion 132. The proximal end portion of the cannula 130 is supported by the grip member 110. Assuming intratumoral administration through use of an endoscope, in particular, administration through use of a gastrointestinal endoscope such as a gastroscope or large intestinal fiberscope, a total length of the cannula 130 preferably is more than or equal to 1.5 m and less than or equal to 2.6 m, and assuming administration through use of a bronchoscope, preferably is more than or equal to 0.6 m and less than or equal to 1.0 m. For smoothly discharging the virus formulation through the puncture needle 131 in response to an operation performed by the surgeon under these conditions while minimizing the virus formulation to be left in the inner cavity of the cannula 130, the inventors of the present invention carried out trial and error to obtain a result that the diameter of the inner cavity of the cannula 130 preferably was more than or equal to 0.2 mm and less than or equal to 1.0 mm.

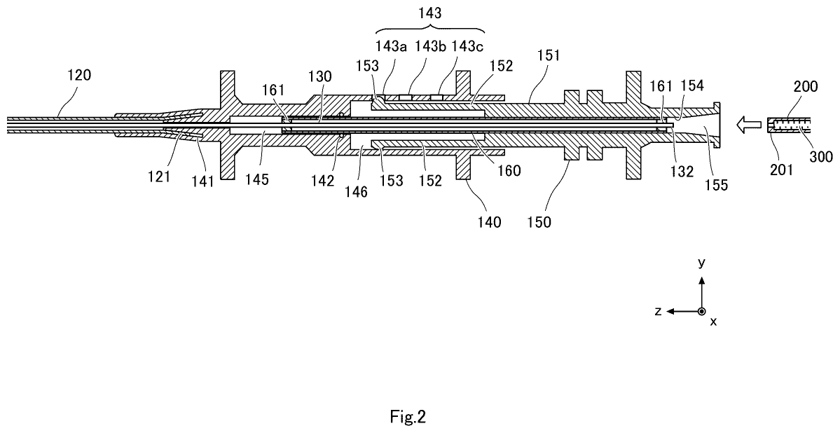

FIG. 2 is a cross-sectional view of the puncture needle unit 100. Specifically, FIG. 2 is a cross-sectional view cut along a yz-plane including a central line of the cannula 130 shown in FIG. 1 and mainly shows a condition around the grip member 110.

In the present embodiment, the grip member 110 is mainly formed from a fixing section 140, a movable section 150, and a holder tube 160. The fixing section 140 supports a sheath proximal end portion 121 which is an end on the proximal end side of the sheath 120. Specifically, a press-fit slit 141 for press-fitting the sheath proximal end portion 121 is provided on a distal end side of the fixing section 140, and the sheath proximal end portion 121 is fixed to the fixing section 140 by being press-fitted into the press-fit slit 141. The cannula 130 inserted into the sheath 120 extends further from the sheath proximal end portion 121 to the proximal end side through the fixing section 140.

The movable section 150 is configured to support the proximal end portion of the cannula 130 and to be capable of moving forward/backward with respect to the fixing section 140 in an insertion direction of the cannula 130 into the sheath 120. Specifically, first, the proximal end portion of the cannula 130 is inserted through the holder tube 160 made of stainless steel and is fixed to the holder tube 160 with both ends of the cannula 130 sealed with rubber stoppers 161. The holder tube 160 is fixed to the movable section 150 by being fit-inserted through a fit-insertion hole 154 provided as part of an inner cavity of the movable section 150. By interposing the holder tube 160 as described, the cannula 130 is reliably fixed to the movable section 150 without any excessive load imposed on the thin cannula 130.

The holder tube 160 reaches a first inner cylinder portion 145 provided as part of an inner cavity of the fixing section 140 and has its outer circumferential surface guided by an inner circumferential surface of the first inner cylinder portion 145, enabling the movable section 150 to move forward/backward with respect to the fixing section 140. An O-ring 142 is attached to the inner circumferential surface of the first inner cylinder portion 145. The O-ring 142 is loosely fitted over the outer circumferential surface of the holder tube 160 to prevent improper forward/backward movement of the movable section 150.

A proximal end side of the fit-insertion hole 154 communicates with a syringe storage portion 155, and an end of the syringe storage portion 155 forms a proximal end side opening of the movable section 150. The syringe 200 in which a virus formulation 300 is held is inserted into the syringe storage portion 155 through the proximal end side opening and attached thereto. A connection base 201 of the syringe 200 is attached to the attachment portion 132 of the cannula 130 provided so as to protrude slightly from the holder tube 160. When pressed by a plunger not shown, the virus formulation 300 is directly supplied to the inner cavity of the cannula 130 via the attachment portion 132.

The movable section 150 has two extended portions 152 extended toward the distal end side from a body portion 151 having the fit-insertion hole 154 provided therein. The extended portions 152 are thin-plate-like cantilevers and are provided on their distal ends with projections 153 protruding in an outer circumferential direction. The fixing section 140 has a second inner cylinder portion 146 communicating with the first inner cylinder portion 145 and having a radius larger than the radius of the first inner cylinder portion 145. An end of the second inner cylinder portion 146 forms a proximal end side opening of the fixing section 140. The second inner cylinder portion 146 has an inner diameter formed to be somewhat larger than an outer diameter of the body portion 151 of the movable section 150, and when the movable section 150 moves forward/backward with respect to the fixing section 140, the projections 153 come into contact with an inner circumferential surface of the second inner cylinder portion 146.

An inner wall of the second inner cylinder portion 146 has three slide holes 143 (a first hole 143a, a second hole 143b, and a third hole 143c) provided in a direction in which one of the extended portions 152 is extended. When one of the projections 153 that is opposed to the slide holes 143 is fitted in any of the slide holes 143 in the case where the movable section 150 is moved forward/backward (slid) with respect to the fixing section 140, the movable section 150 is temporarily fixed at that position. When the surgeon applies a certain force, the projection 153 gets out of the slide hole 143 to allow the movable section 150 to be moved forward/backward again. Since the cannula 130 is fixed to the movable section 150 with the holder tube 160 interposed therebetween, the cannula 130 is moved forward/backward inside the sheath 120 fixed to the fixing section 140 when the movable section 150 is moved forward/backward with respect to the fixing section 140.

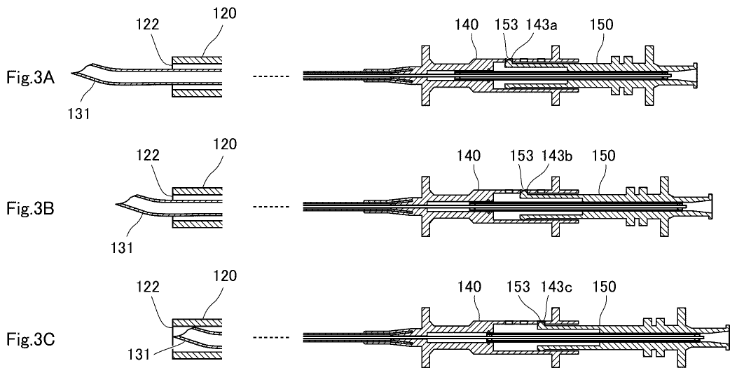

FIG. 3 is a diagram describing forward/backward movement of the puncture needle 131 in response to an operation of the grip member 110. Specifically, FIG. 3 shows conditions of the movable section 150 moved forward/backward with respect to the fixing section 140 to positions different from one another and conditions of the puncture needle 131 at those times, respectively. The conditions of the puncture needle 131 are shown in a larger size than the conditions of the grip member 110.

FIG. 3A shows a condition when the projection 153 is fitted in the first hole 143a among the slide holes 143. At this time, the puncture needle 131 protrudes largely from the open distal end portion 122 of the sheath 120. In the present embodiment, the puncture needle 131 is designed to have a protruding amount of 10 mm.

FIG. 3B shows a condition when the projection 153 is fitted in the second hole 143b among the slide holes 143. At this time, the puncture needle 131 protrudes a little from the open distal end portion 122 of the sheath 120. In the present embodiment, the puncture needle 131 is designed to have a protruding amount of 5 mm.

FIG. 3C shows a condition when the projection 153 is fitted in the third hole 143c among the slide holes 143. At this time, the puncture needle 131 does not protrude from the open distal end portion 122 of the sheath 120 and is in a state stored in the sheath 120.

As described, the slide holes 143 and the projection 153 provided for the extended portions 152 function as a protruding amount adjustment mechanism that changes in a stepwise manner the protruding amount of the puncture needle 131 protruding from the open distal end portion 122. As described above, for example, by setting the protruding amount at two levels of 5 mm and 10 mm, an appropriate protruding amount can be selected in accordance with a state of a tumor site to which the virus formulation is to be administered. The protruding amount adjustment mechanism allows a surgeon operating the puncture needle unit to intuitively recognize the protruding amount of the puncture needle 131. Since the puncture needle 131 can be stored in the sheath 120, the puncture needle 131 can be prevented from being caught when the sheath 120 is inserted through the forceps channel of the endoscope, and surrounding tissues in the vicinity of the tumor site can be prevented from being damaged, for example.

In the present embodiment, the puncture needle 131 is slightly curved with respect to the insertion direction of the cannula 130 into the sheath 120 as shown in the drawing. Such a curve facilitates insertion of the cannula 130 through the sheath 120.

The puncture needle unit 100 described above is assumed to be applied to therapies for digestive system tumors and respiratory tumors, but is applicable not only to digestive system tumors and respiratory tumors but also to other tumors in a case where a virus therapy is effective. Inner cavity radii of the sheath 120 and the cannula 130, the protruding amount of the puncture needle 131, and the like should only be adjusted as appropriate depending on each intended use.

### REFERENCE SIGNS LIST

100 puncture needle unit
110 grip member
120 sheath
121 sheath proximal end portion
122 open distal end portion
130 cannula
131 puncture needle
132 attachment portion
140 fixing section
141 press-fit slit
142 O-ring
143 slide hole
145 first inner cylinder portion
146 second inner cylinder portion
150 movable section
151 body portion
152 extended portion
153 projection
154 fit-insertion hole
155 syringe storage portion
160 holder tube
161 rubber stopper
200 syringe
201 connection base
300 virus formulation

1. An endoscopic puncture needle unit for administering a virus formulation to a tumor site, comprising:
a sheath to be inserted through a forceps channel of an endoscope;
a cannula to be inserted into the sheath and having a distal end portion processed into a puncture needle; and
a grip member configured to support a sheath proximal end portion which is a proximal end portion of the sheath and a cannula proximal end portion which is a proximal end portion of the cannula, a syringe configured to hold the virus formulation being attachable to the grip member, wherein
the cannula is formed integrally from an attachment portion to be directly attached to a connection base of the syringe to the puncture needle configured to discharge the virus formulation.

2. The endoscopic puncture needle unit according to claim 1, wherein
the endoscopic puncture needle unit is used in conjunction with a gastrointestinal endoscope, and
the cannula has an inner cavity diameter of more than or equal to 0.2 mm and less than or equal to 1.0 mm and a total length of more than or equal to 1.5 m and less than or equal to 2.6 m.

3. The endoscopic puncture needle unit according to claim 1, wherein
the endoscopic puncture needle unit is used in conjunction with a bronchoscope, and
the cannula has an inner cavity diameter of more than or equal to 0.2 mm and less than or equal to 1.0 mm and a total length of more than or equal to 0.6 m and less than or equal to 1.0 m.

4. The endoscopic puncture needle unit according to claim 1, wherein a material of the cannula is stainless steel.

5. The endoscopic puncture needle unit according to claim 4, wherein a material of the sheath is fluororesin.

6. The endoscopic puncture needle unit according to claim 1, wherein
the grip member has a fixing section configured to support the sheath proximal end portion and a movable section configured to support the cannula proximal end portion and to be capable of moving forward/backward with respect to the fixing section in an insertion direction of the cannula into the sheath, and
the puncture needle protrudes from an open distal end portion of the sheath or is stored in the open distal end portion when the movable section is moved forward/backward with respect to the fixing section.

7. The endoscopic puncture needle unit according to claim 6, wherein the grip member has a protruding amount adjustment mechanism configured to change in a stepwise manner a protruding amount of the puncture needle configured to protrude from the open distal end portion.

8. The endoscopic puncture needle unit according to claim 6, wherein the puncture needle is curved with respect to the insertion direction.
